SEQUENCE LISTING (19 sequences)

SEQ ID NO: 1 - DNA, 5509 bp, Artificial sequence
  Expression vector combining elements from multiple organisms
gatctggatccgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcttatgcatgctatactgtttttggcttggggtctatacacccccgcttcctcatgttataggtgatggtatagcttagcctataggtgtgggttattgaccattattgaccactcccctattggtgacgatactttccattactaatccataacatggctctttgccacaactctctttattggctatatgccaatacactgtccttcagagactgacacggactctgtatttttacaggatggggtctcatttattatttacaaattcacatatacaacaccaccgtccccagtgcccgcagtttttattaaacataacgtgggatctccacgcgaatctcgggtacgtgttccggacatgggctcttctccggtagcggcggagcttctacatccgagccctgctcccatgcctccagcgactcatggtcgctcggcagctccttgctcctaacagtggaggccagacttaggcacagcacgatgcccaccaccaccagtgtgccgcacaaggccgtggcggtagggtatgtgtctgaaaatgagctcggggagcgggcttgcaccgctgacgcatttggaagacttaaggcagcggcagaagaagatgcaggcagctgagttgttgtgttctgataagagtcagaggtaactcccgttgcggtgctgttaacggtggagggcagtgtagtctgagcagtactcgttgctgccgcgcgcgccaccagacataatagctgacagactaacagactgttcctttccatgggtcttttctgcagtcaccgtccttgacacgaagcttgcagttacgaattcgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagctggccgctgggatccctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgaccagcttaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgacgcccgccccacgacccgcagcgcccgaccgaaaggagcgcacgaccccatgcatcgaacttgtttattgcagcttataatggttacaaataaagcaatagcatcacaaatttcacaaataaagcatttttttcactgcattctagttgtggtttgtccaaactcatcaatgtatcttatcatgtctggatcgccggcgacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaa

SEQ ID NO: 2 - DNA, 5237 bp, Artificial sequence
  Expression vector combining elements from multiple organisms
gatctggatccttggagctaagccagcaatggtagagggaagattctgcacgtcccttccaggcggcctccccgtcaccaccccccccaacccgccccgaccggagctgagagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatccgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgaccttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaagcttgcagttacgaattcgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagctggccgctgggatccctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgaccagcttaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgacgcccgccccacgacccgcagcgcccgaccgaaaggagcgcacgaccccatgcatcgaacttgtttattgcagcttataatggttacaaataaagcaatagcatcacaaatttcacaaataaagcatttttttcactgcattctagttgtggtttgtccaaactcatcaatgtatcttatcatgtctggatcgccggcgacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaa

SEQ ID NO: 3 - DNA, 5931 bp, Artificial sequence
  Expression vector combining elements from multiple organisms
gatctggatccgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcttatgcatgctatactgtttttggcttggggtctatacacccccgcttcctcatgttataggtgatggtatagcttagcctataggtgtgggttattgaccattattgaccactcccctattggtgacgatactttccattactaatccataacatggctctttgccacaactctctttattggctatatgccaatacactgtccttcagagactgacacggactctgtatttttacaggatggggtctcatttattatttacaaattcacatatacaacaccaccgtccccagtgcccgcagtttttattaaacataacgtgggatctccacgcgaatctcgggtacgtgttccggacatgggctcttctccggtagcggcggagcttctacatccgagccctgctcccatgcctccagcgactcatggtcgctcggcagctccttgctcctaacagtggaggccagacttaggcacagcacgatgcccaccaccaccagtgtgccgcacaaggccgtggcggtagggtatgtgtctgaaaatgagctcggggagcgggcttgcaccgctgacgcatttggaagacttaaggcagcggcagaagaagatgcaggcagctgagttgttgtgttctgataagagtcagaggtaactcccgttgcggtgctgttaacggtggagggcagtgtagtctgagcagtactcgttgctgccgcgcgcgccaccagacataatagctgacagactaacagactgttcctttccatgggtcttttctgcagtcaccgtccttgacacgaagcttgcagttacgaattcgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagctggccgctgggatccagctttttgcaaaagcctaggcctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagctggttctttccgcctcagaaggtacctaaccaagttcctctttcagaggttatttcaggccaccttccaccatggccacctcagcaagttcccacttgaacaaaaacatcaagcaaatgtacttgtgcctgccccagggtgagaaagtccaagccatgtatatctgggttgatggtactggagaaggactgcgctgcaaaacccgcaccctggactgtgagcccaagtgtgtagaagagttacctgagtggaattttgatggctctagtacctttcagtctgagggctccaacagtgacatgtatctcagccctgttgccatgtttcgggaccccttccgcagagatcccaacaagctggtgttctgtgaagttttcaagtacaaccggaagcctgcagagaccaatttaaggcactcgtgtaaacggataatggacatggtgagcaaccagcacccctggtttggaatggaacaggagtatactctgatgggaacagatgggcacccttttggttggccttccaatggctttcctgggccccaaggtccgtattactgtggtgtgggcgcagacaaagcctatggcagggatatcgtggaggctcactaccgcgcctgcttgtatgctggggtcaagattacaggaacaaatgctgaggtcatgcctgcccagtgggaatttcaaataggaccctgtgaaggaatccgcatgggagatcatctctgggtggcccgtttcatcttgcatcgagtatgtgaagactttggggtaatagcaacctttgaccccaagcccattcctgggaactggaatggtgcaggctgccataccaactttagcaccaaggccatgcgggaggagaatggtctgaagcacatcgaggaggccatcgagaaactaagcaagcggcaccggtaccacattcgagcctacgatcccaaggggggcctggacaatgcccgtcgtctgactgggttccacgaaacgtccaacatcaacgacttttctgctggtgtcgccaatcgcagtgccagcatccgcattccccggactgtcggccaggagaagaaaggttactttgaagaccgccgcccctctgccaattgtgacccctttgcagtgacagaagccatcgtccgcacatgccttctcaatgagactggcgacgagcccttccaatacaaaaactaacgcccgccccacgacccgcagcgcccgaccgaaaggagcgcacgaccccatgcatcgaacttgtttattgcagcttataatggttacaaataaagcaatagcatcacaaatttcacaaataaagcatttttttcactgcattctagttgtggtttgtccaaactcatcaatgtatcttatcatgtctggatcgccggcgacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaa

SEQ ID NO: 4 - DNA, 5659 bp, Artificial sequence
  Expression vector combining elements from multiple organisms
gatctggatccttggagctaagccagcaatggtagagggaagattctgcacgtcccttccaggcggcctccccgtcaccaccccccccaacccgccccgaccggagctgagagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatccgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgaccttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaagcttgcagttacgaattcgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagctggccgctgggatccagctttttgcaaaagcctaggcctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagctggttctttccgcctcagaaggtacctaaccaagttcctctttcagaggttatttcaggccaccttccaccatggccacctcagcaagttcccacttgaacaaaaacatcaagcaaatgtacttgtgcctgccccagggtgagaaagtccaagccatgtatatctgggttgatggtactggagaaggactgcgctgcaaaacccgcaccctggactgtgagcccaagtgtgtagaagagttacctgagtggaattttgatggctctagtacctttcagtctgagggctccaacagtgacatgtatctcagccctgttgccatgtttcgggaccccttccgcagagatcccaacaagctggtgttctgtgaagttttcaagtacaaccggaagcctgcagagaccaatttaaggcactcgtgtaaacggataatggacatggtgagcaaccagcacccctggtttggaatggaacaggagtatactctgatgggaacagatgggcacccttttggttggccttccaatggctttcctgggccccaaggtccgtattactgtggtgtgggcgcagacaaagcctatggcagggatatcgtggaggctcactaccgcgcctgcttgtatgctggggtcaagattacaggaacaaatgctgaggtcatgcctgcccagtgggaatttcaaataggaccctgtgaaggaatccgcatgggagatcatctctgggtggcccgtttcatcttgcatcgagtatgtgaagactttggggtaatagcaacctttgaccccaagcccattcctgggaactggaatggtgcaggctgccataccaactttagcaccaaggccatgcgggaggagaatggtctgaagcacatcgaggaggccatcgagaaactaagcaagcggcaccggtaccacattcgagcctacgatcccaaggggggcctggacaatgcccgtcgtctgactgggttccacgaaacgtccaacatcaacgacttttctgctggtgtcgccaatcgcagtgccagcatccgcattccccggactgtcggccaggagaagaaaggttactttgaagaccgccgcccctctgccaattgtgacccctttgcagtgacagaagccatcgtccgcacatgccttctcaatgagactggcgacgagcccttccaatacaaaaactaacgcccgccccacgacccgcagcgcccgaccgaaaggagcgcacgaccccatgcatcgaacttgtttattgcagcttataatggttacaaataaagcaatagcatcacaaatttcacaaataaagcatttttttcactgcattctagttgtggtttgtccaaactcatcaatgtatcttatcatgtctggatcgccggcgacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaa

SEQ ID NO: 5 - DNA, 683 bp, Human cytomegalovirus
attggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccg

SEQ ID NO: 6 - DNA, 121 bp, Human cytomegalovirus
tcagatcgcctggagacgccatccacgctgttttgacctccatagaagacaccgggaccgatccagcctccgcggccgggaacggtgcattggaacgcggattccccgtgccaagagtgac

SEQ ID NO: 7 - DNA, 827 bp, Human cytomegalovirus
gtaagtaccgcctatagagtctataggcccacccccttggcttcttatgcatgctatactgtttttggcttggggtctatacacccccgcttcctcatgttataggtgatggtatagcttagcctataggtgtgggttattgaccattattgaccactcccctattggtgacgatactttccattactaatccataacatggctctttgccacaactctctttattggctatatgccaatacactgtccttcagagactgacacggactctgtatttttacaggatggggtctcatttattatttacaaattcacatatacaacaccaccgtccccagtgcccgcagtttttattaaacataacgtgggatctccacgcgaatctcgggtacgtgttccggacatgggctcttctccggtagcggcggagcttctacatccgagccctgctcccatgcctccagcgactcatggtcgctcggcagctccttgctcctaacagtggaggccagacttaggcacagcacgatgcccaccaccaccagtgtgccgcacaaggccgtggcggtagggtatgtgtctgaaaatgagctcggggagcgggcttgcaccgctgacgcatttggaagacttaaggcagcggcagaagaagatgcaggcagctgagttgttgtgttctgataagagtcagaggtaactcccgttgcggtgctgttaacggtggagggcagtgtagtctgagcagtactcgttgctgccgcgcgcgccaccagacataatagctgacagactaacagactgttcctttccatgggtcttttctgcag

SEQ ID NO: 8 - DNA, 17 bp, Human cytomegalovirus
tcaccgtccttgacacg

SEQ ID NO: 9 - DNA, 449 bp, Homo sapiens
ttggagctaagccagcaatggtagagggaagattctgcacgtcccttccaggcggcctccccgtcaccaccccccccaacccgccccgaccggagctgagagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgtt

SEQ ID NO: 10 - DNA, 33 bp, Homo sapiens
ctttttcgcaacgggtttgccgccagaacacag

SEQ ID NO: 11 - DNA, 943 bp, Artificial sequence
  Intron sequence modified from naturally occurring intron sequence in human EF-1 alpha gene
gtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatccgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgaccttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcag

SEQ ID NO: 12 - DNA, 272 bp, Unknown
  Herpes simplex virus TKpA sequence
gggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagc

SEQ ID NO: 13 - DNA, 508 bp, Unknown
  Murine PGK promoter sequence
ctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacc

SEQ ID NO: 14 - DNA, 600 bp, Unknown
  Bacteria puromycin resistance gene
atgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctga

SEQ ID NO: 15 - DNA, 135 bp, Simian Virus 40
aacttgtttattgcagcttataatggttacaaataaagcaatagcatcacaaatttcacaaataaagcatttttttcactgcattctagttgtggtttgtccaaactcatcaatgtatcttatcatgtctggatc

SEQ ID NO: 16 - DNA, 342 bp, Simian Virus 40
agctttttgcaaaagcctaggcctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccac

SEQ ID NO: 17 - DNA, 1122 bp, Unknown
  cDNA sequence for a hamster glutamine synthetase gene
atggccacctcagcaagttcccacttgaacaaaaacatcaagcaaatgtacttgtgcctgccccagggtgagaaagtccaagccatgtatatctgggttgatggtactggagaaggactgcgctgcaaaacccgcaccctggactgtgagcccaagtgtgtagaagagttacctgagtggaattttgatggctctagtacctttcagtctgagggctccaacagtgacatgtatctcagccctgttgccatgtttcgggaccccttccgcagagatcccaacaagctggtgttctgtgaagttttcaagtacaaccggaagcctgcagagaccaatttaaggcactcgtgtaaacggataatggacatggtgagcaaccagcacccctggtttggaatggaacaggagtatactctgatgggaacagatgggcacccttttggttggccttccaatggctttcctgggccccaaggtccgtattactgtggtgtgggcgcagacaaagcctatggcagggatatcgtggaggctcactaccgcgcctgcttgtatgctggggtcaagattacaggaacaaatgctgaggtcatgcctgcccagtgggaatttcaaataggaccctgtgaaggaatccgcatgggagatcatctctgggtggcccgtttcatcttgcatcgagtatgtgaagactttggggtaatagcaacctttgaccccaagcccattcctgggaactggaatggtgcaggctgccataccaactttagcaccaaggccatgcgggaggagaatggtctgaagcacatcgaggaggccatcgagaaactaagcaagcggcaccggtaccacattcgagcctacgatcccaaggggggcctggacaatgcccgtcgtctgactgggttccacgaaacgtccaacatcaacgacttttctgctggtgtcgccaatcgcagtgccagcatccgcattccccggactgtcggccaggagaagaaaggttactttgaagaccgccgcccctctgccaattgtgacccctttgcagtgacagaagccatcgtccgcacatgccttctcaatgagactggcgacgagcccttccaatacaaaaactaa

SEQ ID NO: 18 - DNA, 861 bp, Unknown
  Bacteria ampicillin resistance gene
atgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaa

SEQ ID NO: 19 - DNA, 615 bp, Unknown
  Origin of replication for bacteria plasmid pUC19
aaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcg

(19) United States
(12) Patent Application Publication
(10) Pub. No.: US 2016/0194660 A1
(43) Pub. Date: Jul. 7, 2016

(54) EXPRESSION VECTORS FOR RECOMBINANT PROTEIN PRODUCTION IN MAMMALIAN CELLS

(71) Applicants: Jianxin YE, Kenilworth, NJ (US); MERCK SHARP & DOHME CORP., Rahway, NJ (US)

(72) Inventor: Jianxin YE, East Brunswick, NJ (US)

(73) Assignee: Merck Sharp & Dohme Corp., Rahway, NJ (US)

(21) Appl. No.: 14/653,872

(22) PCT Filed: Dec. 18, 2013
(86) PCT No.: PCT/US13/75922

§ 371 Date: Jun. 19, 2015

Related U.S. Application Data
(60) Provisional application No. 61/740,629, filed on Dec. 21, 2012.

(51) Int. Cl. C12N 15/85; C12N 15/70; C07K 16/00; C12N 15/64; C12P 21/00

(52) U.S. Cl. CPC C12N 15/85; C12N 15/64; C12P 21/00; C07K 16/00; C12N 15/70; C12N 2800/107; C12N 2800/101; C07K 2317/14; C12N 2800/50; C12N 2820/55

(57) ABSTRACT

The invention provides expression vectors that support high levels of polypeptide expression in mammalian cells. The vectors contain at least one expression cassette for a target polypeptide; an expression cassette for a eukaryotic selectable marker protein; an expression cassette for a bacterial selectable marker protein, and a bacterial plasmid origin of replication.

### FIELD OF THE INVENTION

The present invention relates to the expression of polypeptides in mammalian cells, and in particular to expression vectors that support high levels of polypeptide expression in such cells.

### BACKGROUND OF THE INVENTION

Most biopharmaceuticals are produced in mammalian cells transfected with an expression vector that drives constitutive and high level expression of the recombinant protein (See, e.g., Wurm, F. M., Nature Biotech. 22:1393-1398 (2004)). Chinese hamster ovary (CHO) cells are one of the most commonly used cell lines in the commercial production of recombinant protein therapeutics, including monoclonal antibodies. Increased demand for protein therapeutics has bolstered efforts to augment cell line productivity through improvements in expression technology and optimization of process conditions. (See, e.g., Wurm, supra; Birch, J. R. & Racher, A. J., Adv. Drug Delivery Rev. 58:671-685 (2006)).

A well-designed expression vector is the first step toward achieving high production of recombinant proteins. (See, e.g., Ludwig, D. L., BioProcess International 4:S14-S23 (2006)). Expression vectors generally include a number of components: one or more polypeptide expression cassettes, one or more selectable markers, and elements to allow replication of the vector in prokaryotic cells. A typical polypeptide expression cassette comprises a transcription enhancer, promoter, a nucleotide sequence encoding the target polypeptide, and a polyadenylation signal. Additional components that are sometimes included in the expression casset are a 5′ untranslated region and intron. In general, selection of the different components to include in an expression vector will impact target polypeptide expression in mammalian host cells, and it is typically unpredictable if any new combination of components will support high levels of polypeptide expression.

### SUMMARY OF THE INVENTION

The present invention provides expression vectors that support high level of expression of recombinant proteins in mammalian cells and are replicable in bacterial cells. Host cells comprising these expression vectors, and their use in producing recombinant proteins, also form part of the present invention.

In one embodiment, an expression vector of the invention comprises at least one expression cassette for a target polypeptide, an expression cassette for a eukaryotic selection marker, an expression cassette for a bacterial selection marker, and a bacterial plasmid origin of replication. These elements may be arranged in a variety of orders relative to each other in the vector. The expression vector is typically provided as a circular double-stranded DNA molecule, but in some embodiments, the expression vector may be produced as a linear double-stranded DNA molecule.

The target polypeptide expression cassette comprises a promoter operably linked to an insertion site for a nucleotide sequence encoding the target polypeptide and a first polyadenylation (polyA) signal. In some embodiments, the promoter is a construct comprising the promoter sequence, the first 5′ untranslated region (UTR1), the first intron, and a portion of the second 5′ untranslated region (UTR2) from the immediate early (IE) gene of a cytomegalovirus (CMV) or an elongation factor 1 alpha (EF-1 alpha) gene of a mammal. Some preferred embodiments further comprise the nucleotide sequence encoding the target polypeptide.

The expression vector of the invention also comprises an expression cassette for a eukaryotic selection marker, which comprises a second promoter operably linked to a nucleotide sequence encoding a puromycin resistance protein or a glutamine synthetase (GS) protein and to a second polyA signal. The identity of the promoter for driving expression of the eukaryotic selection marker depends on the identity of the protein to be expressed. If the selection marker is a puromycin resistance protein, then the promoter shares substantial identity with, or is identical to, the promoter of a mammalian 3-phosphoglycerate kinase (PGK) gene. Alternatively, if the selection marker is a GS protein, then the promoter shares substantial identity with, or is identical to, the promoter of a simian virus 40 (SV40) late gene.

The first and second polyA signals in the target polypeptide and the eukaryotic selection marker expression cassettes, respectively, may consist of the same or different polyA sequences, and each shares substantial identity with, or is identical to, the poly A signal in the thymidine kinase (TK) gene of Herpes Simplex Virus (HSV TKpA) or the poly A signal in the early gene for Simian Virus 40 (SV40 pA). In one preferred embodiment, the first polyA signal in the target polypeptide expression cassette is a TKpA sequence and the second polyA signal in the eukaryotic selection marker expression construct is an SV40 pA sequence.

In another embodiment, the invention provides an expression vector that is capable of expressing two target polypeptides, and which comprises an expression cassette for a first target polypeptide, an expression cassette for a second target polypeptide, an expression cassette for a eukaryotic selection marker, an expression cassette for a bacterial selection marker, and a bacterial plasmid origin of replication. Such vectors are useful to express proteins that are composed of two different polypeptide chains, e.g., monoclonal antibodies. The individual components of such dimeric expression vectors may be arranged in a variety of orders in the vector, yet have the same nucleotide sequences and are present in the same combinations as described above or elsewhere herein.

Another aspect of the invention is a recombinant host cell which comprises a mammalian cell transfected with any of the expression vector embodiments described above or elsewhere herein. The expression vector may be integrated into the chromosomal DNA of the recombinant cell or not integrated. Furthermore, the recombinant cell can contain more than one copy of the expression vector, for example, two or more copies per cell. The host cell is useful for producing a target polypeptide by a method which comprises culturing the cell under conditions in which the polypeptide is expressed, and recovering the polypeptide from the culture.

In a still further aspect, the invention provides a recombinant host cell which comprises a bacterial cell transformed with any of the expression vector embodiments described above or elsewhere herein. The recombinant bacterial cell is useful for propogating the expression vector by a method of propogating an expression vector, which comprises culturing the cell under conditions in which the expression vector is replicated, and recovering the expression vector from the culture.

### BRIEF DESCRIPTION OF THE FIGURES

FIG. 1 illustrates the structure of the PJY21 expression vector, with FIG. 1A showing the arrangement of various functional elements and restriction enzyme sites in the vector and FIGS. 1B and 1C showing the complete nucleotide sequence of the vector (SEQ ID NO:1).

FIG. 2 illustrates the structure of the PJY22 expression vector, with FIG. 2A showing the arrangement of various functional elements and restriction enzyme sites in the vector and the FIGS. 2B and 2C showing the complete nucleotide sequence of the vector (SEQ ID NO:2).

FIG. 3 illustrates the structure of the PJY41 expression vector, with FIG. 3A showing the arrangement of various functional elements and restriction enzyme sites in the vector and FIGS. 3B and 3C showing the complete nucleotide sequence of the vector (SEQ ID NO:3).

FIG. 4 illustrates the structure of the PJY42 expression vector, with FIG. 4A showing the arrangement of various functional elements and restriction enzyme sites in the vector and FIGS. 4B and 4C showing the complete nucleotide sequence of the vector (SEQ ID NO:4).

FIG. 5 illustrates the structure of a preferred embodiment of an antibody expression vector of the invention in which two identical tandem expression cassettes separately express the light and heavy chains of a monoclonal antibody.

FIG. 6 illustrates the varying ability of four different expression vectors to generate large numbers of transfected CHOK1 clones that express high expression levels of a model monoclonal antibody.

FIG. 7 illustrates expression levels of a model monoclonal antibody after a 14 day fed-batch culture of multiple clones stably transfected with one of three expression vectors.

### DETAILED DESCRIPTION OF THE INVENTION

### I. General

The present invention is not to be limited in scope by the specific embodiments described herein. Indeed, various modifications of the invention in addition those described herein will become apparent to those skilled in the art from the foregoing description. Such modifications are intended to fall within the scope of the appended claims.

Patents, patent applications, publications, product descriptions, and protocols are cited throughout this application, the disclosure of such documents are incorporated herein by reference in their entirety for all purposes, and to the same extent as if each individual document was specifically and individually indicated to be incorporated by reference.

### II. Molecular Biology and Definitions

In accordance with the present invention there may be employed conventional molecular biology, microbiology, and recombinant DNA techniques within the skill of the art. Such techniques are explained fully in the literature. See, e.g., Sambrook, Fritsch & Maniatis, Molecular Cloning: A Laboratory Manual, Second Edition (1989) Cold Spring Harbor Laboratory Press, Cold Spring Harbor, N.Y. (herein “Sambrook, et al., 1989”); DNA Cloning: A Practical Approach, Volumes I and II (D. N. Glover ed. 1985); Oligonucleotide Synthesis (M. J. Gait ed. 1984); Nucleic Acid Hybridization (B. D. Hames & S. J. Higgins eds. (1985)); Transcription And Translation (B. D. Hames & S. J. Higgins, eds. (1984)); Animal Cell Culture (R. I. Freshney, ed. (1986)); Immobilized Cells And Enzymes (IRL Press, (1986)); B. Perbal, A Practical Guide To Molecular Cloning (1984); F. M. Ausubel, et al. (eds.), Current Protocols in Molecular Biology, John Wiley & Sons, Inc. (1994).

So that the invention may be more readily understood, certain technical and scientific terms are specifically defined below. Unless specifically defined elsewhere in this specification, all other technical and scientific terms use herein have the meaning that would be commonly understood by one of ordinary skill in the art to which this invention belongs when used in similar contexts as used herein.

As used herein, including the appended claims, the singular forms of words such as “a,” “an,” and “the,” include their corresponding plural references unless the context clearly dictates otherwise.

“About” when used to modify a numerically defined parameter, e.g., the length of a polynucleotide discussed herein, means that the parameter may vary by as much as 10% below or above the stated numerical value for that parameter. For example, a polynucleotide of about 100 bases may vary between 90 and 110 bases.

A “coding sequence” is a nucleotide sequence that encodes a biological product of interest (e.g., an RNA, polypeptide, protein, or enzyme) and when expressed, results in production of the product. A coding sequence is “under the control of”, “functionally associated with” or “operably linked to” or “operably associated with” transcriptional or translational control sequences in a cell when the sequences direct RNA polymerase mediated transcription of the coding sequence into RNA, e.g., mRNA, which then may be trans-RNA spliced (if it contains introns) and, optionally, translated into a protein encoded by the coding sequence.

“Consists essentially of” and variations such as “consist essentially of” or “consisting essentially of” as used throughout the specification and claims, indicate the inclusion of any recited elements or group of elements, and the optional inclusion of other elements, of similar or different nature than the recited elements, which do not materially change the basic or novel properties of the specified dosage regimen, method, or composition.

“Express” and “expression” mean allowing or causing the information in a gene or coding sequence, e.g., an RNA or DNA, to become manifest; for example, producing a protein by activating the cellular functions involved in transcription and translation of a corresponding gene. A DNA sequence can be expressed in or by a cell to form an “expression product” such as an RNA (e.g., mRNA) or a protein. The expression product itself may also be said to be “expressed” by the cell.

“Expression vector” or “expression construct” means a vehicle (e.g., a plasmid) by which a polynucleotide comprising regulatory sequences operably linked to a coding sequence can be introduced into a host cell where the coding sequence is expressed using the transcription and translation machinery of the host cell.

“Host cell” includes any cell of any organism that is manipulated by a human for the purpose of producing an expression product encoded by an expression vector introduced into the host cell. A “recombinant mammalian host cell” refers to a mammalian cell that comprises a heterologous expression vector, which may or may not be integrated into a host cell chromosome.

“Hybridization conditions” means the combination of temperature and composition of the hybridization solution that are used in a hybridization reaction between at least two polynucleotides (see Sambrook, et al., supra). Hybridization solution typically includes different strengths of SSC, which is 0.15M NaCl and 0.015M Na-citrate. Examples of low stringency hybridization conditions are: (1) 55° C., 5×SSC, 0.1% SDS, 0.25% milk, no formamide; and (2) 30% formamide, 5×SSC, 0.5% SDS. Moderate stringency hybridization conditions are 55° C., 40% formamide, and 5× or 6×SSC. High stringency hybridization conditions employ 50% formamide, 5× or 6×SSC and temperatures from about 55° C. to about 68° C. (i.e., 55° C., 56° C. 57° C., 58° C., 59° C., 60° C., 61° C., 62° C., 63° C., 64° C., 65° C., 66° C., 67° C. or 68° C.).

“Isolated” is typically used to reflect the purification status of a biological molecule such as RNA, DNA, oligonucleotide, polynucleotide or protein, and in such context means the molecule is substantially free of other biological molecules such as nucleic acids, proteins, lipids, carbohydrates, or other material such as cellular debris and growth media. Generally, the term “isolated” is not intended to refer to a complete absence of other biological molecules or material or to an absence of water, buffers, or salts, unless they are present in amounts that substantially interfere with the methods of the present invention.

“Nucleic acid” refers to a single- or double-stranded polymer of bases attached to a sugar phosphate backbone, and includes DNA and RNA molecules.

“Oligonucleotide” refers to a nucleic acid that is usually between 5 and 100 contiguous nucleotides in length, and most frequently between 10-50, 10-40, 10-30, 10-25, 10-20, 15-50, 15-40, 15-30, 15-25, 15-20, 20-50, 20-40, 20-30 or 20-25 contiguous nucleotides in length.

“Polynucleotide” refers to a nucleic acid that is 13 or more contiguous nucleotides in length.

“Promoter” or “promoter sequence” is, in an embodiment of the invention, a DNA regulatory region capable of binding an RNA polymerase in a cell (e.g., directly or through other promoter-bound proteins or substances) and initiating transcription of a coding sequence. Within the promoter sequence may be found a transcription initiation site (conveniently defined, for example, by mapping with nuclease S1), as well as protein binding domains (consensus sequences) responsible for the binding of RNA polymerase as well an enhancer element.

“Promoter activity” refers to a physical measurement of the strength of the promoter.

“Selectable marker” is a protein which allows the specific selection of cells which express this protein by the addition of a corresponding selecting agent to the culture medium.

### III. Preferred Embodiments of the Invention

The present invention provides, in part, an expression vector comprising a bacterial origin of replication and three separate expression cassettes: a first cassette for expressing a target polypeptide, a second cassette for expressing a selectable marker protein that allows the selection of eukaryotic cells stably transfected with the vector, an a third cassette for expressing a selectable marker protein that allows the selection of bacteria cells transformed with the expression vector.

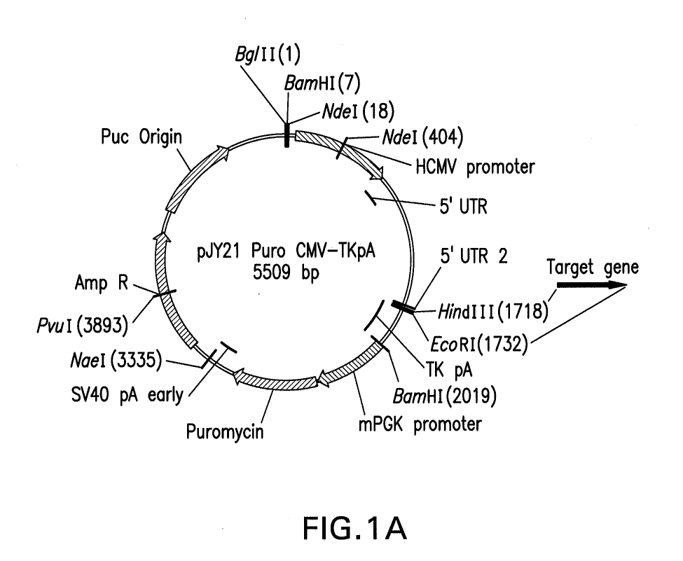

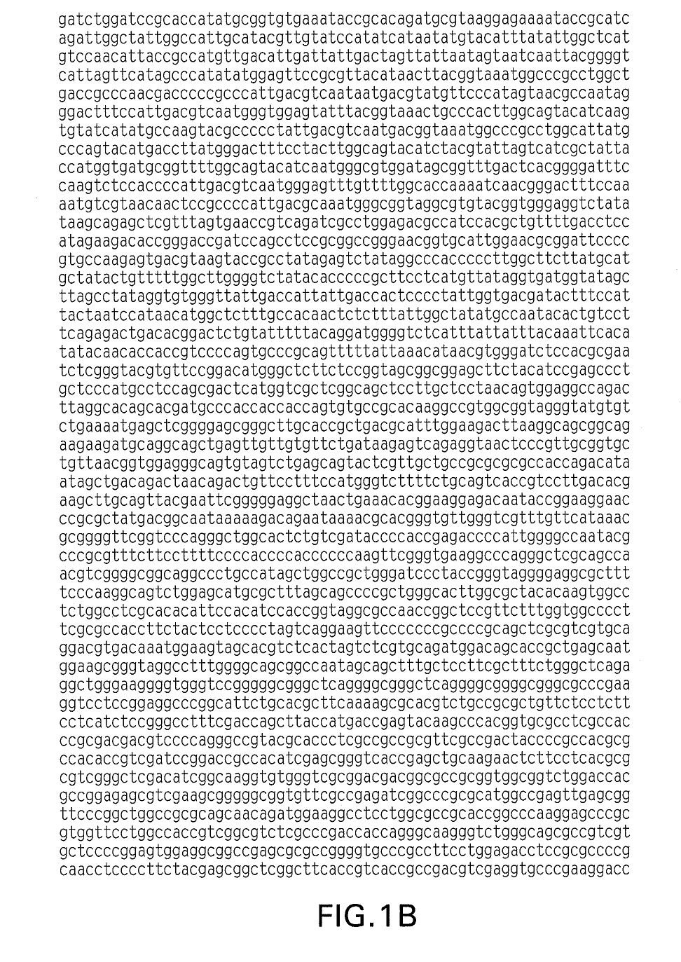

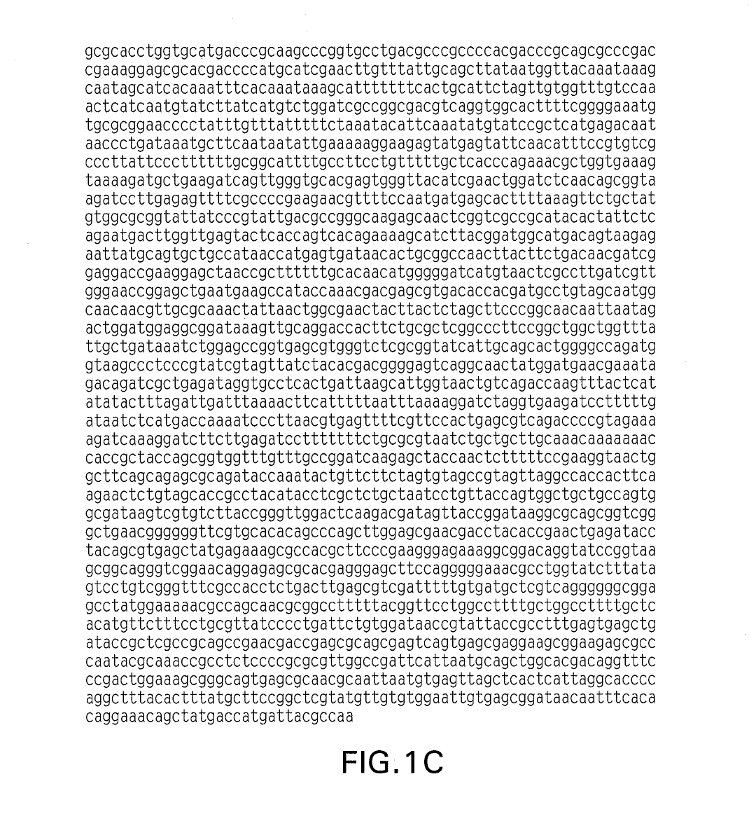

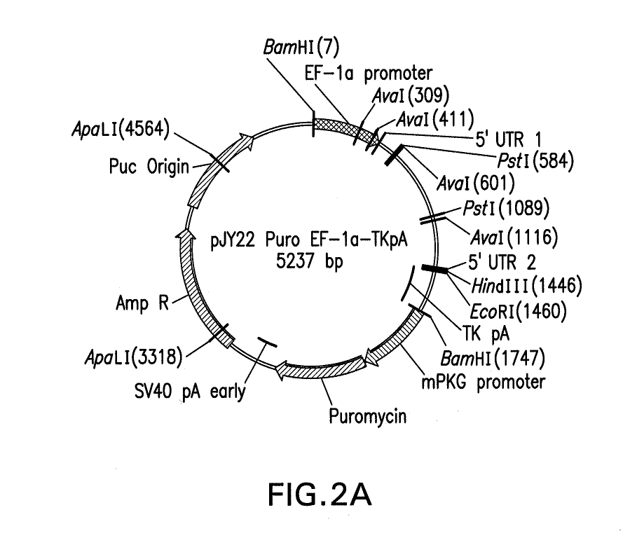

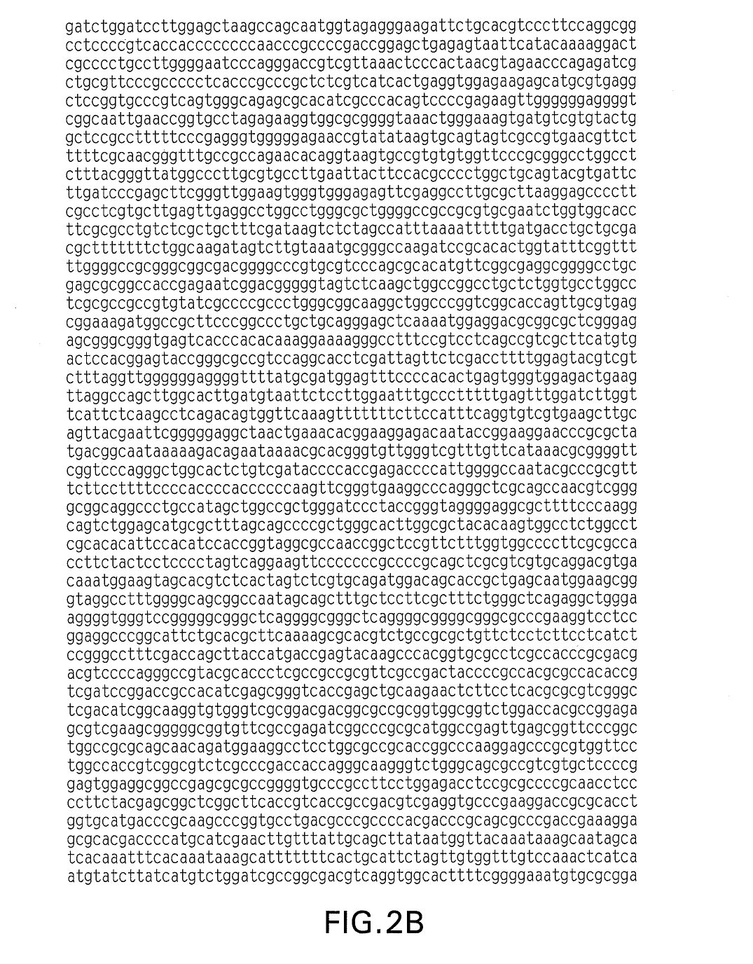

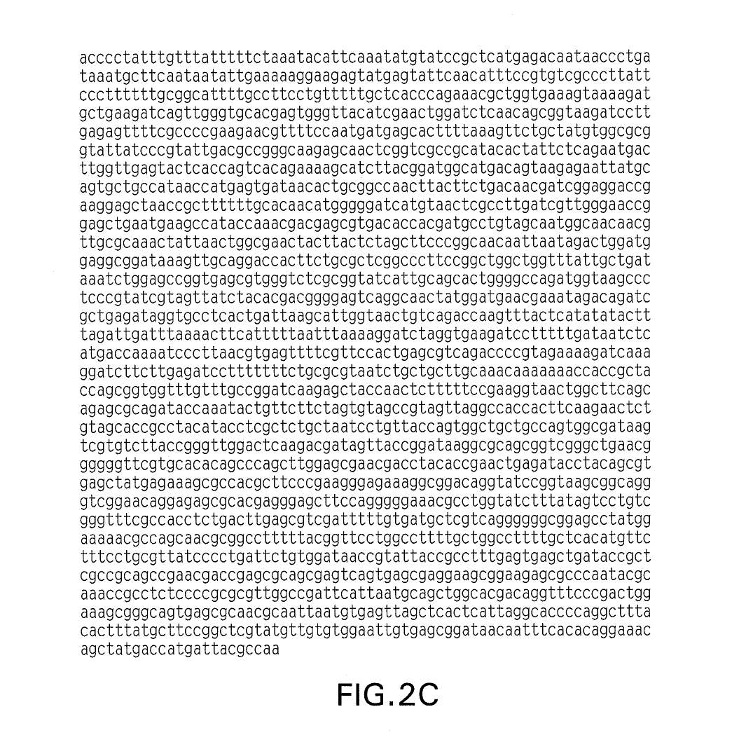

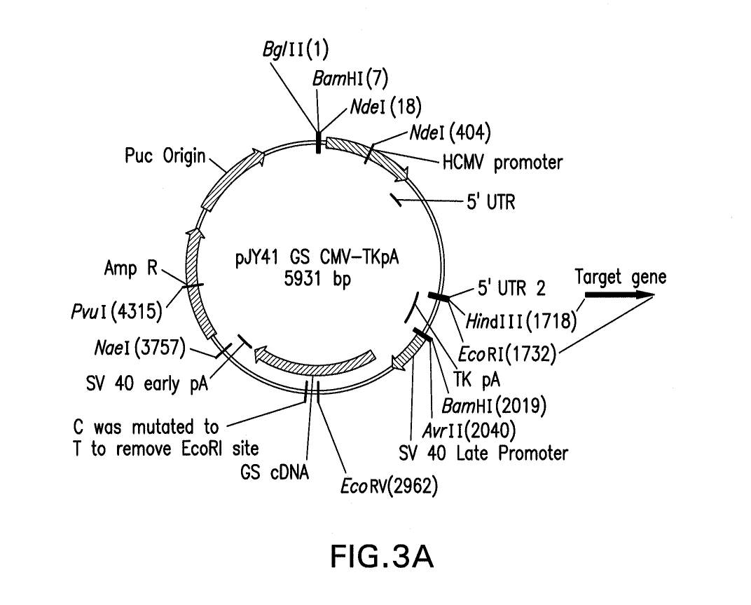

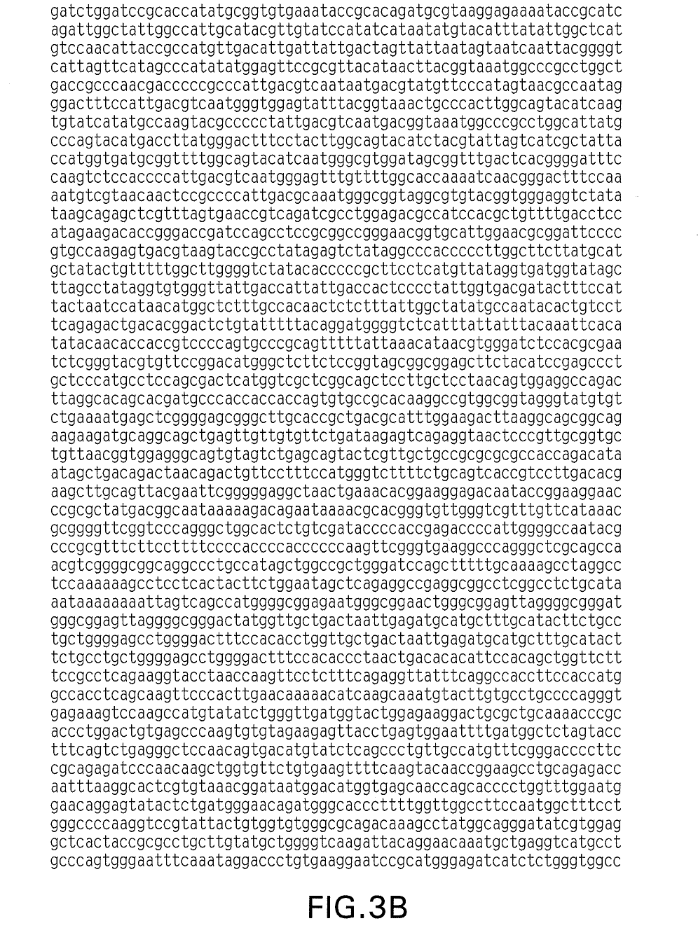

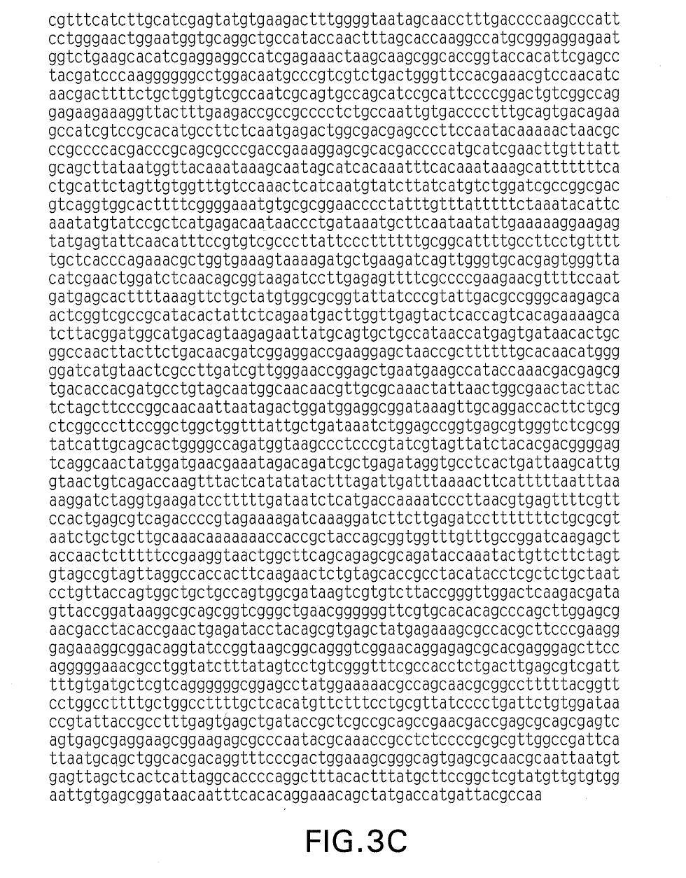

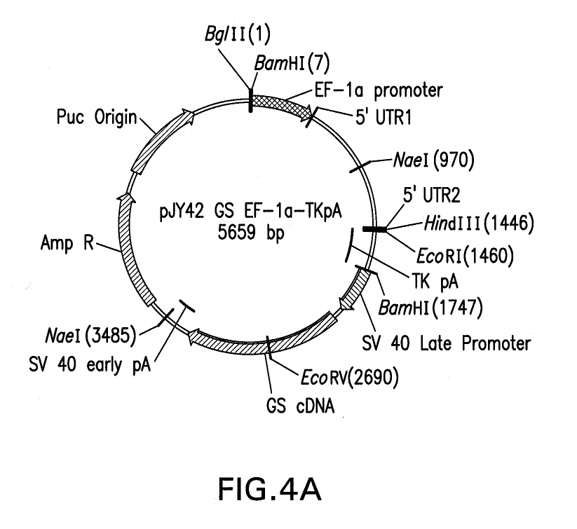

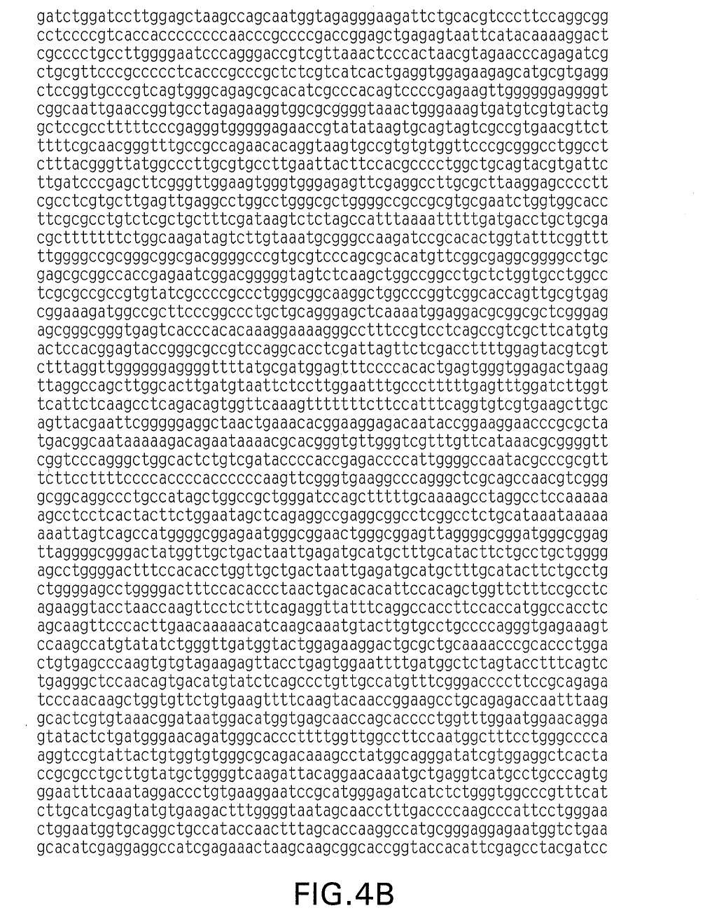

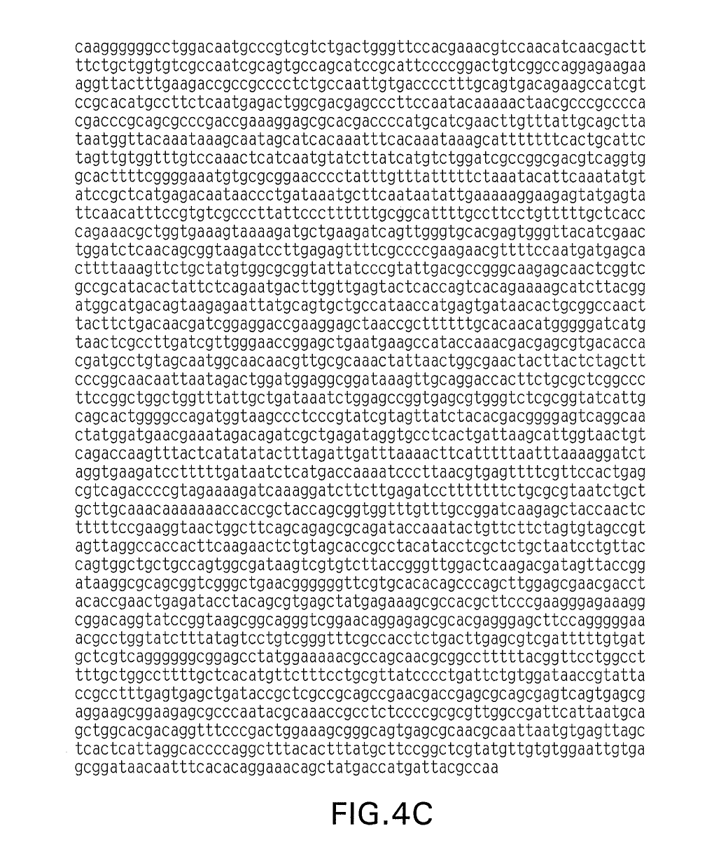

The three expression cassettes may be arranged in the vector in any order relative to each other. In some embodiments, the order is as shown in FIGS. 1-4, i.e., the target polypeptide cassette is upstream of the eukaryotic selection marker cassette, which is upstream of the bacteria selection marker cassette, which is located between the origin of replication and the target polypeptide cassette. In other embodiments, the eukaryotic selection marker cassette is upstream of the target polypeptide expression cassette.

Similarly, the relative positions of the promoter and polyA expression control elements in one or more of the expression cassettes may vary such that the direction of transcription is not shared by all three cassettes. For example, the direction of transcription of the nucleotide sequence encoding the eukaryotic selection marker may be the opposite of the transcription direction employed in the target polypeptide expression cassette.

In some embodiments, the first expression cassette comprises a site for inserting a nucleotide sequence that encodes the target polypeptide downstream and in operable linkage to the promoter. The insertion site typically comprises at least one restriction enzyme (RE) recognition sequence, and may include two or more RE sequences to form a multiple cloning site (MCS). In a particularly preferred embodiment, the insertion site consists of the recognition sequences for the Hind III and EcoRI enzymes. Cleavage of the circular vector with these two enzymes creates a linear vector to which a nucleotide sequence encoding the polypeptide with appropriate “sticky” ends may be attached.

Target polypeptides that may be expressed by an expression vector of the invention include, but are not limited to, therapeutic polypeptides such as adhesion molecules, antibody light and/or heavy chains, cytokines, enzymes, lymphokines, and receptors. Expression of the target polypeptide is driven by a CMV promoter construct or an EF-1 alpha promoter construct.

In some embodiments, the expression vector is adapted to express two target polypeptides, such as the individual polypeptide chains in a heterodimeric protein. Such embodiments contain two target polypeptide expression cassettes, which are identical in composition with the exception of having different nucleotide sequences encoding the different target polypeptides. It is contemplated that the two polypeptide expression cassettes may be separated by one or more of the other elements of the vector. Preferably, the two target polypeptide expression cassettes are arranged in tandem in the vector.

In some preferred embodiments, the expression vector is adapted to express a monoclonal antibody (mAb), with one of the target polypeptide expression cassettes encoding the light chain of the mAb, and the other target polypeptide expression cassette encoding the heavy chain of the mAb. The light chain expression cassette may be upstream of downstream of the heavy chain expression cassette. Preferably, the light chain expression cassette is upstream of the downstream expression cassette.

In some preferred embodiments, the nucleotide sequence of the CMV promoter construct is at least 90% identical to the human CMV contiguous sequence formed from SEQ ID NOs 5, 6, 7 and 8, i.e., nucleotides 69-1,716 of SEQ ID NO:1. The nucleotide sequence of a preferred CMV promoter construct is at least 95%, 96%, 97%, 98% or 99% identical to nucleotides 69-1,716 of SEQ ID NO:1.

In other preferred embodiments, the EF-1 alpha promoter construct is at least 90% identical to the human EF-1 alpha contiguous sequence formed from SEQ ID NOs 9, 10, 11 and 12, i.e., nucleotides 12-1,444 of SEQ ID NO:2. The nucleotide sequence of a preferred EF-1 alpha promoter construct is at least 95%, 96%, 97%, 98% or 99% identical to 12-1,444 of SEQ ID NO:2.

The eukaryotic selectable marker expressed by the second expression cassette is a puromycin resistance protein or a GS protein. Expression of the puromycin resistance protein allows cells transfected with a vector of the invention to grow in media containing puromycin. Alternatively, cells transfected with a vector of the invention that expresses the GS protein are capable of growing in glutamine free media, and selection pressure for such cells may be increased by including the GS inhibitor methionine sulfoximine (MSX) in the media.

In some preferred embodiments, the nucleotide sequence encoding the puromycin resistance protein is at least 95%, 96%, 97%, 98%, or 99% identical to the murine nucleotide sequence of SEQ ID NO:15. Most preferably, the nucleotide sequence encoding the puromycin resistance protein consists of SEQ ID NO:14.

The promoter used to drive expression of the puromycin resistance protein is a PGK promoter. In some preferred embodiments, the PGK promoter is a nucleotide sequence that is at 95%, 96%, 97%, 98%, or 99% identical to the murine PGK promoter sequence of SEQ ID NO:13. Most preferably, the PGK promoter consists of SEQ ID NO:13.

In some preferred embodiments, the nucleotide sequence encoding the GS protein is at least 95%, 96%, 97%, 98%, or 99% identical to the hamster cDNA sequence of SEQ ID NO:17. Most preferably, the GS encoding sequence consists of SEQ ID NO:17.

The promoter used to drive expression of the GS protein is an SV40 late promoter. In some preferred embodiments, the SV40 later promoter is a nucleotide sequence that is at 95%, 96%, 97%, 98%, or 99% identical to the SV40 later promoter sequence of SEQ ID NO:16. Most preferably, the SV40 late promoter consists of SEQ ID NO:16.

Another transcription control element present in each of the first and second expression cassettes is a polyA signal, which is a polyA signal from a thymidine kinase (TK) gene (TKpA) or a simian virus 40 (SV40) early gene (SV40 pA). In particularly preferred embodiments, the polyA signal in the first expression cassette is a TKpA signal and the polyA signal in the second expression cassette is an SV40 pA signal.

In some preferred embodiments, the TKpA signal consists of a nucleotide sequence that is at least 95%, 96%, 97%, 98%, or 99% identical to the herpes simplex virus (HSV) TKpA sequence of SEQ ID NO:12. Most preferably, the TKpA signal consists of SEQ ID NO:12.

In other preferred embodiments, the SV40 pA signal consists of a nucleotide sequence that is at least 95%, 96%, 97%, 98%, or 99% identical to the SV40 pA sequence of SEQ ID NO:15. Most preferably, the SV40 pA signal consists of SEQ ID NO:15.

The third expression cassette comprises a nucleotide sequence that encodes a bacterial selection marker. Nonlimiting examples of selectable markers useful in the vectors of the invention are proteins that confer resistance of bacterial cells to an antibiotic, e.g., ampicillin, tetracycline, hygromycin, kanamycin, blasticidin and the like. In a preferred embodiment, the antibiotic is ampicillin and the encoding nucleotide sequence is at least 95%, 96%, 97%, 98%, or 99% identical to the coding sequence set forth in SEQ ID NO:18.

A bacterial plasmid origin of replication is also present in expression vectors of the invention to facilitate preparation of large quantities of the vector in bacteria cells. Nonlimiting examples of plasmid replication origins include pUC origins derived from pBR322. In preferred embodiments, the origin of replication is a nucleotide sequence that is at least 95%, 96%, 97%, 98%, or 99% identical to the pUC19 origin of replication sequence shown in SEQ ID NO:19. Most preferably, the origin of replication in an expression vector of the invention consists of SEQ ID NO:19.

In some embodiments, the origin of replication is located between the bacterial selection marker and the target polypeptide expression cassette. Other arrangements for these two vector elements are contemplated, including e.g., one in which the target polypeptide expression cassette is located between the origin of replication and the expression cassette for the bacterial selection marker.

In any of the embodiments of the invention described herein, when a first nucleotide sequence is defined in terms of identity to a second, reference nucleotide sequence, the first sequence is identical in length to the reference sequence, but has at least one nucleotide position in which a different nucleotide has been substituted for the reference nucleotide.

The invention also contemplates that the nucleotide sequence for an individual vector component of the invention may be obtained from a different species than the species listed in Example 1 for the corresponding vector component. For example, a species variant of the human EF-1 alpha promoter could consist of the nucleotide sequence of the promoter in the mouse or hamster EF-1 alpha gene. Similarly, a species variant of the HSV TKpA signal could consist of the nucleotide sequence of the TKpA signal for a different herpes virus. Preferably, a polynucleotide or oligonucleotide consisting of a species variant nucleotide sequence will hybridize under high stringency conditions to a polynucleotide or oligonucleotide consisting of the reference nucleotide sequence.

Embodiments that do comprise a nucleotide sequence that encodes a target polypeptide are useful for producing the target polypeptide in mammalian cell culture by any method well known in the art. In one embodiment, the method comprises transfecting a mammalian host cell with the vector and culturing the transfected cell under selection conditions in which the target polypeptide is expressed. The expression vector may be introduced into a mammalian host cell by any of several methods known in the art, such as, for example, the calcium phosphate coprecipitation method as described by Graham and Van der Eb, Virology, 52: 546 (1978), nuclear injection, protoplast fusion, electroporation, liposomal transformation and DEAE-Dextran transformation. The expression vector may be linearized to enhance integration into the host cell genome. The linearization site should be located at a site in the vector backbone that avoids impact on the expression of the target polypeptide or the eukaryotic selectable marker protein.

Suitable mammalian host cells include hamster cells such as BHK21, BHK RK⁻, CHO, CHO-K1, CHO-DUKX, CHO-DUKX B1 and CHO-DG44 cells or derivatives/descendants of these cell lines. Preferred host cells are CHO-DG44, CHO-DBX11, CHO-DUKX, CHO-K1 and BHK21 cells. Also suitable are myeloma cells from the mouse, preferably NS0 and Sp2/0-AG14 cells and human cell lines such as HEK293 or PER.C6, as well as derivatives/descendants of these mouse and human cell lines.

In embodiments of the invention where the expression vector encodes a target polypeptide, the vector may be integrated into the genomic DNA of a mammalian host cell (e.g., CHO, CHO-K1, CHO-D1 DXB11) to improve stability or may be ectopic (not integrated). In some preferred embodiments, the vector of the present invention is present in the cell at several copies per cell (e.g., 2, 3, 4, 5, 6, 7, 8, 9, 10, 15, 20). Where an expression vector has been integrated into the genomic DNA of the host cell, the copy number of the vector, and, concomitantly, the amount of target polypeptide expressed, can be increased by selecting for cell lines in which the vector sequences have been amplified after integration into the DNA of the host cell.

Any of several cell culture mediums known in the art can be used to propagate mammalian cells expressing a target polypeptide of interest. Several commercially available culture mediums are available. If expressing a polypeptide that is to be used therapeutically, animal-product-free media (e.g., serum-free media (SFM)) is desirable. There are several methods known in the art by which to cells may be adapted to growth in serum-free medium.

Selective conditions in the culture medium will vary depending on the host cell line and selectable markers used. For CHO cells transfected with a vector that expresses a puromycin resistance protein, the media typically contains 7 to 20 micrograms/ml puromycin. When the eukaryotic selectable marker is a GS protein, a glutamine-free media is used to culture transfected CHO cells, and 10-50 micromolar MSX may be added.

### EXAMPLES

These examples are intended to further clarify the present invention and not to limit the invention. Any composition or method, in whole or in part, set forth in the examples form a part of the present invention.

### Example 1

### Construction of Backbone Expression Vectors

Backbone vectors were generated that included various combinations of the following functional components: a target polypeptide expression cassette, a eukaryotic selection marker expression cassette, a bacterial resistance selection marker cassette, and a bacterial origin of replication.

The target gene expression cassette contained a human cytoniegalovirus immediate-early (hCMV IE) promoter construct or human Elongation factor 1-alpha (EF-1α) promoter construct for driving expression of a target protein, a restriction enzyme site for inserting a nucleotide sequence encoding the target protein, and the polyadenylation signal (pA) from the herpes simplex virus (HSV) thymidine kinase gene (HSV TKpA).

Two different eukaryotic selection marker expression cassettes were used: a puromycin resistance expression cassette and a glutamine synthetase (GS) expression cassette. Expression of the puromycin resistance protein was driven by the promoter for the mouse 3-phosphoglycerate kinase (mPGK) gene. In the GS cassette, a Simian virus 40 (SV40) late promoter sequence was operably linked to a hamster GS cDNA sequence. Each eukaryotic selection marker cassette included the SV40 early polyA signal.

The bacterial selection marker cassette included the promoter and encoding sequence from a bacterial ampicillin resistance gene.

The bacterial origin of replication was the replication origin from the pUC19 cloning vector to allow replication in E. coli.

DNA fragments corresponding to each of the above vector elements were chemically synthesized and ligated together to generate the backbone expression vectors shown in FIGS. 1-4. The sequences of the individual backbone vector elements are shown below.

1. hCMV IE Promoter Construct

Promoter Sequence (SEQ ID NO: 5):
attggctattggccattgcatacgttgtatccatatcataatatgtacat
ttatattggctcatgtccaacattaccgccatgttgacattgattattga
ctagttattaatagtaatcaattacggggtcattagttcatagcccatat
atggagttccgcgttacataacttacggtaaatggcccgcctggctgacc
gcccaacgacccccgcccattgacgtcaataatgacgtatgttcccatag
taacgccaatagggactttccattgacgtcaatgggtggagtatttacgg
taaactgcccacttggcagtacatcaagtgtatcatatgccaagtacgcc
ccctattgacgtcaatgacggtaaatggcccgcctggcattatgcccagt
acatgaccttatgggactttcctacttggcagtacatctacgtattagtc
atcgctattaccatggtgatgcggttttggcagtacatcaatgggcgtgg
atagcggtttgactcacggggatttccaagtctccaccccattgacgtca
atgggagtttgttttggcaccaaaatcaacgggactttccaaaatgtcgt
aacaactccgccccattgacgcaaatgggcggtaggcgtgtacggtggga
ggtctatataagcagagctcgtttagtgaaccg
5′ UTR1 Sequence (exon 1 of hCMV IE gene)
(SEQ ID NO: 6):
tcagatcgcctggagacgccatccacgctgttttgacctccatagaagac
accgggaccgatccagcctccgcggccgggaacggtgcattggaacgcgg
attccccgtgccaagagtgac
Intron Sequence (SEQ ID NO: 7):
gtaagtaccgcctatagagtctataggcccacccccttggcttcttatgc
atgctatactgtttttggcttggggtctatacacccccgcttcctcatgt
tataggtgatggtatagcttagcctataggtgtgggttattgaccattat
tgaccactcccctattggtgacgatactttccattactaatccataacat
ggctctttgccacaactctctttattggctatatgccaatacactgtcct
tcagagactgacacggactctgtatttttacaggatggggtctcatttat
tatttacaaattcacatatacaacaccaccgtccccagtgcccgcagttt
ttattaaacataacgtgggatctccacgcgaatctcgggtacgtgttccg
gacatgggctcttctccggtagcggcggagcttctacatccgagccctgc
tcccatgcctccagcgactcatggtcgctcggcagctccttgctcctaac
agtggaggccagacttaggcacagcacgatgcccaccaccaccagtgtgc
cgcacaaggccgtggcggtagggtatgtgtctgaaaatgagctcggggag
cgggcttgcaccgctgacgcatttggaagacttaaggcagcggcagaaga
agatgcaggcagctgagttgttgtgttctgataagagtcagaggtaactc
ccgttgcggtgctgttaacggtggagggcagtgtagtctgagcagtactc
gttgctgccgcgcgcgccaccagacataatagctgacagactaacagact
gttcctttccatgggtcttttctgcag
5′ UTR2 Sequence (only the 5′ part of exon 2 in
the hCMV IE gene) (SEQ ID NO: 8):
tcaccgtccttgacacg

### 2. EF-1α Promoter Construct

Promoter Sequence (SEQ ID NO: 9):
ttggagctaagccagcaatggtagagggaagattctgcacgtcccttcca
ggcggcctccccgtcaccaccccccccaacccgccccgaccggagctgag
agtaattcatacaaaaggactcgcccctgccttggggaatcccagggacc
gtcgttaaactcccactaacgtagaacccagagatcgctgcgttcccgcc
ccctcacccgcccgctctcgtcatcactgaggtggagaagagcatgcgtg
aggctccggtgcccgtcagtgggcagagcgcacatcgcccacagtccccg
agaagttggggggaggggtcggcaattgaaccggtgcctagagaaggtgg
cgcggggtaaactgggaaagtgatgtcgtgtactggctccgcctttttcc
cgagggtgggggagaaccgtatataagtgcagtagtcgccgtgaacgtt
5′ UTR1 Sequence (exon 1 of EF-1α gene)
(SEQ ID NO: 10):
ctttttcgcaacgggtttgccgccagaacacag
Intron Sequence (the underlined nucleotides
represent changes that were made to the
naturally occurring EF-1α sequence: a T to
C substitution to delete a Bgl II site and
a G to C substitution to delete a Xho I site)
(SEQ ID NO: 11):
gtaagtgccgtgtgtggttcccgcgggcctggcctctttacgggttatgg
cccttgcgtgccttgaattacttccacgcccctggctgcagtacgtgatt
cttgatcccgagcttcgggttggaagtgggtgggagagttcgaggccttg
cgcttaaggagccccttcgcctcgtgcttgagttgaggcctggcctgggc
gctggggccgccgcgtgcgaatctggtggcaccttcgcgcctgtctcgct
gctttcgataagtctctagccatttaaaatttttgatgacctgctgcgac
gctttttttctggcaagatagtcttgtaaatgcgggccaagatccgcaca
ctggtatttcggtttttggggccgcgggcggcgacggggcccgtgcgtcc
cagcgcacatgttcggcgaggcggggcctgcgagcgcggccaccgagaat
cggacgggggtagtctcaagctggccggcctgctctggtgcctggcctcg
cgccgccgtgtatcgccccgccctgggcggcaaggctggcccggtcggca
ccagttgcgtgagcggaaagatggccgcttcccggccctgctgcagggag
ctcaaaatggaggacgcggcgctcgggagagcgggcgggtgagtcaccca
cacaaaggaaaagggcctttccgtcctcagccgtcgcttcatgtgactcc
acggagtaccgggcgccgtccaggcacctcgattagttctcgaccttttg
gagtacgtcgtctttaggttggggggaggggttttatgcgatggagtttc
cccacactgagtgggtggagactgaagttaggccagcttggcacttgatg
taattctccttggaatttgccctttttgagtttggatcttggttcattct
caagcctcagacagtggttcaaagtttttttcttccatttcag
5′ UTR2 Sequence (only the 5′ part of exon 2 of
the EF-1α gene):
gtgtcgtg

### 3. HSV TKpA Sequence

(SEQ ID NO: 12):
gggggaggctaactgaaacacggaaggagacaataccggaaggaacccgc
gctatgacggcaataaaaagacagaataaaacgcacgggtgttgggtcgt
ttgttcataaacgcggggttcggtcccagggctggcactctgtcgatacc
ccaccgagaccccattggggccaatacgcccgcgtttcttccttttcccc
accccaccccccaagttcgggtgaaggcccagggctcgcagccaacgtcg
gggcggcaggccctgccatagc

### 4. Puromycin Resistance Expression Cassette:

mPGK Promoter Sequence (SEQ ID NO: 13)
ctaccgggtaggggaggcgcttttcccaaggcagtctggagcatgcgctt
tagcagccccgctgggcacttggcgctacacaagtggcctctggcctcgc
acacattccacatccaccggtaggcgccaaccggctccgttctttggtgg
ccccttcgcgccaccttctactcctcccctagtcaggaagttcccccccg
ccccgcagctcgcgtcgtgcaggacgtgacaaatggaagtagcacgtctc
actagtctcgtgcagatggacagcaccgctgagcaatggaagcgggtagg
cctttggggcagcggccaatagcagctttgctccttcgctttctgggctc
agaggctgggaaggggtgggtccgggggcgggctcaggggcgggctcagg
ggcggggcgggcgcccgaaggtcctccggaggcccggcattctgcacgct
tcaaaagcgcacgtctgccgcgctgttctcctcttcctcatctccgggcc
tttcgacc
Puromycin Resistance Nucleotide Sequence
(SEQ ID NO: 14):
atgaccgagtacaagcccacggtgcgcctcgccacccgcgacgacgtccc
cagggccgtacgcaccctcgccgccgcgttcgccgactaccccgccacgc
gccacaccgtcgatccggaccgccacatcgagcgggtcaccgagctgcaa
gaactcttcctcacgcgcgtcgggctcgacatcggcaaggtgtgggtcgc
ggacgacggcgccgcggtggcggtctggaccacgccggagagcgtcgaag
cgggggcggtgttcgccgagatcggcccgcgcatggccgagttgagcggt
tcccggctggccgcgcagcaacagatggaaggcctcctggcgccgcaccg
gcccaaggagcccgcgtggttcctggccaccgtcggcgtctcgcccgacc
accagggcaagggtctgggcagcgccgtcgtgctccccggagtggaggcg
gccgagcgcgccggggtgcccgccttcctggagacctccgcgccccgcaa
cctccccttctacgagcggctcggcttcaccgtcaccgccgacgtcgagg
tgcccgaaggaccgcgcacctggtgcatgacccgcaagcccggtgcctga
SV40 early pA Sequence (SEQ ID NO: 15):
aacttgtttattgcagcttataatggttacaaataaagcaatagcatcac
aaatttcacaaataaagcatttttttcactgcattctagttgtggtttgt
ccaaactcatcaatgtatcttatcatgtctggatc

### 5. GS Expression Cassette

SV40 Late Promoter Sequence (SEQ ID NO: 16):
Agctttttgcaaaagcctaggcctccaaaaaagcctcctcactacttctg
gaatagctcagaggccgaggcggcctcggcctctgcataaataaaaaaaa
ttagtcagccatggggcggagaatgggcggaactgggcggagttaggggc
gggatgggcggagttaggggcgggactatggttgctgactaattgagatg
catgctttgcatacttctgcctgctggggagcctggggactttccacacc
tggttgctgactaattgagatgcatgctttgcatacttctgcctgctggg
gagcctggggactttccacaccctaactgacacacattccac
Hamster GS cDNA sequence (the underlined nu-
cleotides represent a change that was made to
the naturally occurring GS sequence: a C to T
substitution to delete an EcoRI site)
(SEQ ID NO: 17):
atggccacctcagcaagttcccacttgaacaaaaacatcaagcaaatgta
cttgtgcctgccccagggtgagaaagtccaagccatgtatatctgggttg
atggtactggagaaggactgcgctgcaaaacccgcaccctggactgtgag
cccaagtgtgtagaagagttacctgagtggaattttgatggctctagtac
ctttcagtctgagggctccaacagtgacatgtatctcagccctgttgcca
tgtttcgggaccccttccgcagagatcccaacaagctggtgttctgtgaa
gttttcaagtacaaccggaagcctgcagagaccaatttaaggcactcgtg
taaacggataatggacatggtgagcaaccagcacccctggtttggaatgg
aacaggagtatactctgatgggaacagatgggcacccttttggttggcct
tccaatggctttcctgggccccaaggtccgtattactgtggtgtgggcgc
agacaaagcctatggcagggatatcgtggaggctcactaccgcgcctgct
tgtatgctggggtcaagattacaggaacaaatgctgaggtcatgcctgcc
cagtgggaatttcaaataggaccctgtgaaggaatccgcatgggagatca
tctctgggtggcccgtttcatcttgcatcgagtatgtgaagactttgggg
taatagcaacctttgaccccaagcccattcctgggaactggaatggtgca
ggctgccataccaactttagcaccaaggccatgcgggaggagaatggtct
gaagcacatcgaggaggccatcgagaaactaagcaagcggcaccggtacc
acattcgagcctacgatcccaaggggggcctggacaatgcccgtcgtctg
actgggttccacgaaacgtccaacatcaacgacttttctgctggtgtcgc
caatcgcagtgccagcatccgcattccccggactgtcggccaggagaaga
aaggttactttgaagaccgccgcccctctgccaattgtgaccccttgcag
tgacagaagccatcgtccgcacatgccttctcaatgagactggcgacgag
cccttccaatacaaaaactaa

(SEQ ID NO: 18):
atgagtattcaacatttccgtgtcgcccttattcccttttttgcggcatt
ttgccttcctgtttttgctcacccagaaacgctggtgaaagtaaaagatg
ctgaagatcagttgggtgcacgagtgggttacatcgaactggatctcaac
agcggtaagatccttgagagttttcgccccgaagaacgttttccaatgat
gagcacttttaaagttctgctatgtggcgcggtattatcccgtattgacg
ccgggcaagagcaactcggtcgccgcatacactattctcagaatgacttg
gttgagtactcaccagtcacagaaaagcatcttacggatggcatgacagt
aagagaattatgcagtgctgccataaccatgagtgataacactgcggcca
acttacttctgacaacgatcggaggaccgaaggagctaaccgcttttttg
cacaacatgggggatcatgtaactcgccttgatcgttgggaaccggagct
gaatgaagccataccaaacgacgagcgtgacaccacgatgcctgtagcaa
tggcaacaacgttgcgcaaactattaactggcgaactacttactctagct
tcccggcaacaattaatagactggatggaggcggataaagttgcaggacc
acttctgcgctcggcccttccggctggctggtttattgctgataaatctg
gagccggtgagcgtgggtctcgcggtatcattgcagcactggggccagat
ggtaagccctcccgtatcgtagttatctacacgacggggagtcaggcaac
tatggatgaacgaaatagacagatcgctgagataggtgcctcactgatta
agcattggtaa

### 6. Ampicillin Resistance Gene

7. pUC19 Origin of Replication Sequence

(SEQ ID NO: 19):
aaaggatcttcttgagatcctttttttctgcgcgtaatctgctgcttgca
aacaaaaaaaccaccgctaccagcggtggtttgtttgccggatcaagagc
taccaactctttttccgaaggtaactggcttcagcagagcgcagatacca
aatactgttcttctagtgtagccgtagttaggccaccacttcaagaactc
tgtagcaccgcctacatacctcgctctgctaatcctgttaccagtggctg
ctgccagtggcgataagtcgtgtcttaccgggttggactcaagacgatag
ttaccggataaggcgcagcggtcgggctgaacggggggttcgtgcacaca
gcccagcttggagcgaacgacctacaccgaactgagatacctacagcgtg
agctatgagaaagcgccacgcttcccgaagggagaaaggcggacaggtat
ccggtaagcggcagggtcggaacaggagagcgcacgagggagcttccagg
gggaaacgcctggtatctttatagtcctgtcgggtttcgccacctctgac
ttgagcgtcgatttttgtgatgctcgtcaggggggcggagcctatggaaa
aacgccagcaacgcg

### Example 2

### Antibody Expression in CHO Cells

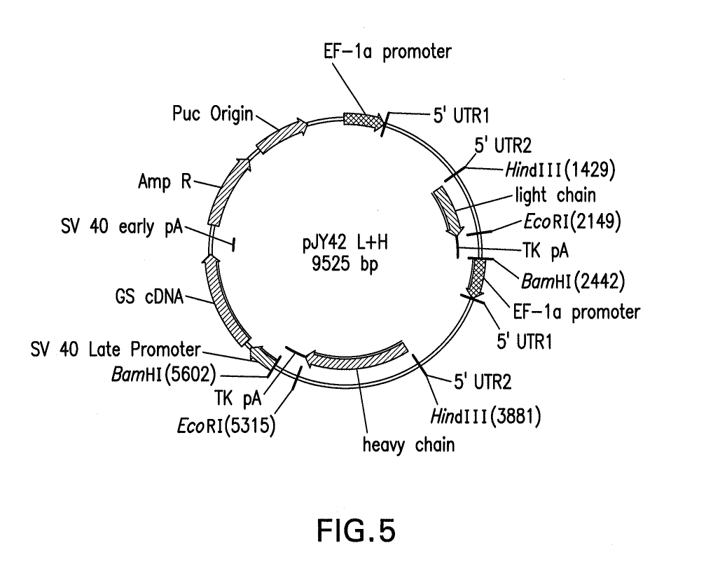

To assess the capability of the vector constructs described in Example 1 to support protein expression in mammalian cells, each of the backbone vectors was modified by inserting a second target gene expression cassette that was identical to the first target gene expression cassette and located immediately downstream of the first cassette. Coding sequences for the light and heavy chains of a model monoclonal antibody were inserted between the HindIII/EcoRI sites of the first and second expression cassettes, respectively, as illustrated in FIG. 5.

Each of the antibody expression vectors were linearized by digestion with Pvu I and transfected by electroporation into wild-type CHOK1 cells that had been adapted in suspension in chemically defined medium. The transfected cells were then seeded in 96-well plates at a seeding density of approximately 10,000 cells per well. After 3 to 4 weeks under appropriate selection, colonies formed in some of the wells. The different vectors produced different number of wells with colony formation. In general, antibody expression vectors with the pJY21 or pJY22 backbone (puromycin selection marker) had 30-50% of wells with cell growth. In contrast, about 6-10% of the wells seeded with the antibody expression vector with the pJY42 backbone (GS selection marker) had cell growth and the pJY41-based vector (GS selection marker) had very few wells with cell growth. Optimization of the selection pressure may improve the cell out-growth.

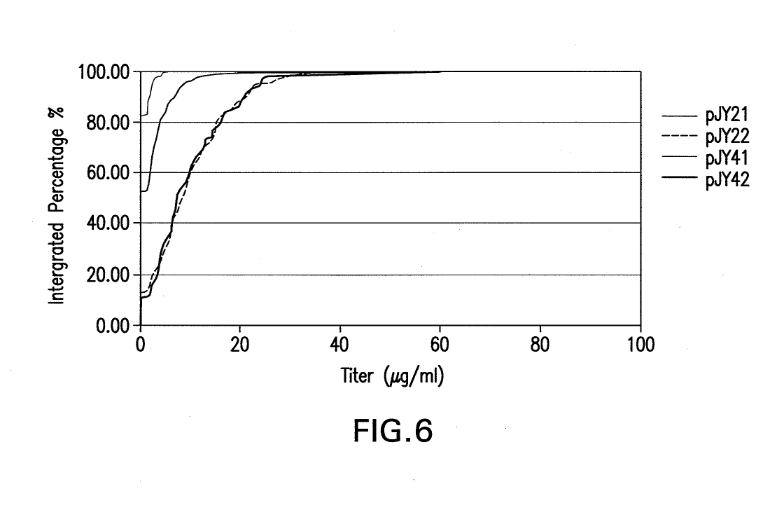

For each transfection, cell culture supernatant was collected from randomly-picked wells that contained a single colony, and Mab expression levels were measured using modified ELISA assay. FIG. 6 shows accumulation rates of clones with different expression levels. Most of the clones containing pJY21, pJY22 or pJY42 have high expression levels, with pJY22 and pJY42 having the highest expression levels. In contrast, very few clones containing the pJY41 vector have high expression levels. These results indicate that the combination of different elements in the target gene expression cassette or the combination of expression cassette elements and eukaryotic selectable marker can have a significant impact on the capability of the vector to support target protein expression.

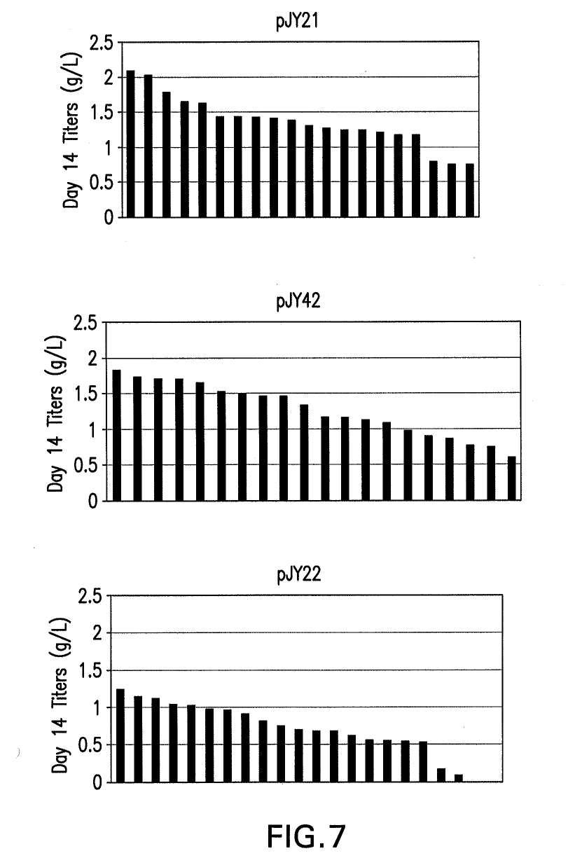

Clones containing the pJY21, pJY22 or pJY42 vectors and which expressed monoclonal antibodies were expanded under appropriate selection, adapted to suspension culture, and then cultured in shake flasks in a 14 day fed-batch process. Cultures were inoculated at 2×10⁵ vc/mL with a working volume of 30-50 milliliters. Cell cultures were fed at ˜5% v/v with an in house formulation of concentrated nutrients containing amino acids, vitamins, nucleosides, and hydrolysates at 2-3 day intervals. Concurrent to feed addition, glucose was fed back to 40 mM. A pJY41 clone was not included in this evaluation due to the very low protein expression levels supported by this vector. Samples were removed from each fed batch culture to measure protein expression by protein A HPLC, and the results are shown in FIG. 7.

The expression vector containing the pJY21 backbone supported the highest expression of the model monoclonal antibody (above 2 g/L), with the pJY42 and pJY22 vectors supporting monoclonal antibody expression to 1.8 g/L, and above 1 g/L, respectively. These results indicate that each of the pJY21, pJY22 or pJY42 vectors can support high levels of protein expression in mammalian cells. Since neither selection pressure nor the fed-batch process used for this evaluation was optimized, it is contemplated that productivity may be improved by optimizing the process conditions.

1-20. (canceled)

21. An expression vector which comprises the following elements:
(a) at least one expression cassette for a first target polypeptide which comprises a first promoter operably linked to an insertion site for a nucleotide sequence encoding the first target polypeptide and a first polyadenylation (pA) signal;
(b) an expression cassette for a eukaryotic selection marker which comprises a second promoter operably linked to a nucleotide sequence encoding the eukaryotic selection marker and to a second pA signal, wherein the eukaryotic selection marker is a puromycin resistance protein or a glutamine synthetase (GS) protein;
(c) an expression cassette for a bacterial selection marker, and
(d) a bacterial plasmid origin of replication,
wherein the first and second pA signals are the same or different and are selected from the group consisting of a thymidine kinase pA (TKpA) sequence and a simian virus 40 (SV40) early pA sequence,
wherein the first promoter is a cytomegalovirus (CMV) promoter construct that is at least 90% identical to nucleotides 69 to 1,716 of SEQ ID NO:1 or an Elongation factor 1-alpha (EF-1 alpha) promoter construct that is at least 90% identical to nucleotides 12-1,444 of SEQ ID NO:2;
wherein the second promoter is a 3-phosphoglycerate kinase (PGK) promoter if the eukaryotic selection marker is a puromycin resistance protein;
wherein the second promoter is a simian virus 40 (SV40) late promoter if the eukaryotic selection marker is a GS protein; and
wherein the eukaryotic selection marker is a puromycin resistance protein if the first promoter is the CMV promoter.

22. The expression vector of claim 21, wherein the CMV promoter construct consists of nucleotides 69 to 1,716 of SEQ ID NO:1, the EF-1 alpha promoter construct consists of nucleotides 12-1,444 of SEQ ID NO:2, and the TKpA sequence is a herpes simplex virus (HSV) TKpA sequence of SEQ ID NO:12.

23. The expression vector of claim 21, wherein the first pA signal is the HSV TK pA sequence of SEQ ID NO:12, the second promoter is the SV40 late promoter sequence of SEQ ID NO:16, the nucleotide sequence encoding the GS protein is the hamster GS cDNA sequence of SEQ ID NO:17 and the second pA signal is the SV40 early pA sequence of SEQ ID NO:15.

24. The expression vector of claim 21, wherein the PGK promoter is the murine PGK promoter sequence of SEQ ID NO:13.

25. The expression vector of claim 21, wherein the bacterial origin of replication is the pUC19 origin of replication sequence of SEQ ID NO:19.

26. The expression vector of claim 21, wherein the insertion site has 5′ and 3′ boundaries defined by 1^{st} and 2^{nd} restriction enzyme recognition sites.

27. The expression vector of claim 26, wherein the restriction enzyme recognition sites are for HindIII and EcoRI.

28. The expression vector of claim 21, which consists of SEQ ID NO:1, SEQ ID NO:2 or SEQ ID NO:4.

29. The expression vector of claim 21, which further comprises a second expression construct for expressing a second target polypeptide, wherein the second expression construct comprises the first promoter operably linked to an insertion site for a nucleotide sequence encoding the second target polypeptide and the first polyadenylation (pA) signal.

30. The expression vector of claim 29, wherein the first target polypeptide is the light chain of a monoclonal antibody and the second target polypeptide is the heavy chain of the monoclonal antibody.

31. The expression vector of claim 21, wherein the vector elements are arranged in the following order: (a), then (b), then (c), and then (d).

32. An expression vector capable of expressing a monoclonal antibody (mAb) in a mammalian host cell, the vector comprising the following elements:
(a) a first expression cassette which comprises a first promoter operably linked to a nucleotide sequence which encodes the light chain of the mAb and a first polyadenylation (pA) signal;
(b) a second expression cassette identical to the first expression cassette except a nucleotide sequence encoding the heavy chain of the mAb is substituted for the nucleotide sequence encoding the mAb light chain;
(c) an expression cassette for a eukaryotic selection marker which comprises a second promoter operably linked to a nucleotide sequence encoding a puromycin resistance protein or a glutamine synthetase (GS) protein and to a second pA signal;
(d) an expression cassette for a bacterial selection marker, and
(e) a bacterial plasmid origin of replication,
wherein the first and second pA signals are the same or different and are selected from the group consisting of a thymidine kinase pA (TKpA) sequence and a simian virus 40 (SV40) early pA sequence,
wherein the first promoter is a cytomegalovirus (CMV) promoter construct that is at least 90% identical to nucleotides 69 to 1,716 of SEQ ID NO:1 or an Elongation factor 1-alpha (EF-1 alpha) promoter construct that is at least 90% identical to nucleotides 12-1,444 of SEQ ID NO:2;
wherein the second promoter is a 3-phosphoglycerate kinase (PGK) promoter if the eukaryotic selection marker is a puromycin resistance protein,
wherein the second promoter is a simian virus 40 (SV40) late promoter if the eukaryotic selection marker is a GS protein, and
wherein the eukaryotic selection marker is puromycin resistance if the first promoter is the HCMV promoter.

33. The expression vector of claim 32, wherein the first promoter is the human CMV promoter construct of nucleotides 69 to 1,716 of SEQ ID NO:1, the first pA signal is the HSV TK pA sequence of SEQ ID NO:12, the second promoter is the murine PGK promoter sequence of SEQ ID NO:13, the nucleotide sequence encoding a puromycin resistance protein is SEQ ID NO:14, the second pA signal is the SV40 early pA sequence of SEQ ID NO:15, the bacterial selection marker is the ampicillin resistance gene sequence of SEQ ID NO:18 and the bacterial origin of replication is the pUC19 origin of replication sequence of SEQ ID NO:19.

34. The expression vector of claim 32, wherein the first promoter is the human EF-1 alpha promoter construct of nucleotide 12 to 1,444 of SEQ ID NO:2, the first pA signal is the HSV TK pA sequence of SEQ ID NO:12, the second promoter is the SV40 late promoter sequence of SEQ ID NO:13, the nucleotide sequence encoding the GS protein is the hamster GS cDNA sequence of SEQ ID NO:17, the second pA signal is the SV40 early pA sequence of SEQ ID NO:15, the bacterial selection marker is the ampicillin resistance gene sequence of SEQ ID NO:18 and the bacterial origin of replication is the pUC19 origin of replication sequence of SEQ ID NO:19.

35. The expression vector of claim 32, wherein the vector elements are arranged in the order: (a), then (b), then (c), then (d) and then (e).

36. A recombinant host cell which comprises a mammalian cell transfected with an expression vector, wherein the vector comprises
(a) at least one expression cassette for a first target polypeptide which comprises a first promoter operably linked to an insertion site for a nucleotide sequence encoding the first target polypeptide and a first polyadenylation (pA) signal;
(b) an expression cassette for a eukaryotic selection marker which comprises a second promoter operably linked to a nucleotide sequence encoding the eukaryotic selection marker and to a second pA signal, wherein the eukaryotic selection marker is a puromycin resistance protein or a glutamine synthetase (GS) protein;
(c) an expression cassette for a bacterial selection marker, and
(d) a bacterial plasmid origin of replication,
wherein the first and second pA signals are the same or different and are selected from the group consisting of a thymidine kinase pA (TKpA) sequence and a simian virus 40 (SV40) early pA sequence,
wherein the first promoter is a cytomegalovirus (CMV) promoter construct that is at least 90% identical to nucleotides 69 to 1,716 of SEQ ID NO:1 or an Elongation factor 1-alpha (EF-1 alpha) promoter construct that is at least 90% identical to nucleotides 12-1,444 of SEQ ID NO:2;
wherein the second promoter is a 3-phosphoglycerate kinase (PGK) promoter if the eukaryotic selection marker is a puromycin resistance protein;
wherein the second promoter is a simian virus 40 (SV40) late promoter if the eukaryotic selection marker is a GS protein; and
wherein the eukaryotic selection marker is a puromycin resistance protein if the first promoter is the CMV promoter.

37. The recombinant host cell of claim 36, wherein the mammalian cell is a CHO K1 cell.

38. A method of producing a polypeptide, comprising providing a recombinant host cell, culturing the cell under conditions in which the polypeptide is expressed, and recovering the polypeptide from the culture,
wherein the recombinant host cell comprises a mammalian cell transfected with an expression vector, and wherein the vector comprises:
(a) at least one expression cassette for a first target polypeptide which comprises a first promoter operably linked to an insertion site for a nucleotide sequence encoding the first target polypeptide and a first polyadenylation (pA) signal;
(b) an expression cassette for a eukaryotic selection marker which comprises a second promoter operably linked to a nucleotide sequence encoding the eukaryotic selection marker and to a second pA signal, wherein the eukaryotic selection marker is a puromycin resistance protein or a glutamine synthetase (GS) protein;
(c) an expression cassette for a bacterial selection marker, and
(d) a bacterial plasmid origin of replication,
wherein the first and second pA signals are the same or different and are selected from the group consisting of a thymidine kinase pA (TKpA) sequence and a simian virus 40 (SV40) early pA sequence,
wherein the first promoter is a cytomegalovirus (CMV) promoter construct that is at least 90% identical to nucleotides 69 to 1,716 of SEQ ID NO:1 or an Elongation factor 1-alpha (EF-1 alpha) promoter construct that is at least 90% identical to nucleotides 12-1,444 of SEQ ID NO:2;
wherein the second promoter is a 3-phosphoglycerate kinase (PGK) promoter if the eukaryotic selection marker is a puromycin resistance protein;
wherein the second promoter is a simian virus 40 (SV40) late promoter if the eukaryotic selection marker is a GS protein; and
wherein the eukaryotic selection marker is a puromycin resistance protein if the first promoter is the CMV promoter.

39. A recombinant host cell which comprises a bacterial cell transformed with an expression vector, wherein the vector comprises:
(a) at least one expression cassette for a first target polypeptide which comprises a first promoter operably linked to an insertion site for a nucleotide sequence encoding the first target polypeptide and a first polyadenylation (pA) signal;
(b) an expression cassette for a eukaryotic selection marker which comprises a second promoter operably linked to a nucleotide sequence encoding the eukaryotic selection marker and to a second pA signal, wherein the eukaryotic selection marker is a puromycin resistance protein or a glutamine synthetase (GS) protein;
(c) an expression cassette for a bacterial selection marker, and
(d) a bacterial plasmid origin of replication,
wherein the first and second pA signals are the same or different and are selected from the group consisting of a thymidine kinase pA (TKpA) sequence and a simian virus 40 (SV40) early pA sequence,
wherein the first promoter is a cytomegalovirus (CMV) promoter construct that is at least 90% identical to nucleotides 69 to 1,716 of SEQ ID NO:1 or an Elongation factor 1-alpha (EF-1 alpha) promoter construct that is at least 90% identical to nucleotides 12-1,444 of SEQ ID NO:2;
wherein the second promoter is a 3-phosphoglycerate kinase (PGK) promoter if the eukaryotic selection marker is a puromycin resistance protein;
wherein the second promoter is a simian virus 40 (SV40) late promoter if the eukaryotic selection marker is a GS protein; and
wherein the eukaryotic selection marker is a puromycin resistance protein if the first promoter is the CMV promoter.

40. A method of propogating an expression vector, comprising providing a recombinant host cell, culturing the cell under conditions in which the expression vector is replicated, and recovering the expression vector from the culture, wherein the recombinant host cell comprises a bacterial cell transformed with an expression vector, wherein the vector comprises:
(a) at least one expression cassette for a first target polypeptide which comprises a first promoter operably linked to an insertion site for a nucleotide sequence encoding the first target polypeptide and a first polyadenylation (pA) signal;
(b) an expression cassette for a eukaryotic selection marker which comprises a second promoter operably linked to a nucleotide sequence encoding the eukaryotic selection marker and to a second pA signal, wherein the eukaryotic selection marker is a puromycin resistance protein or a glutamine synthetase (GS) protein;
(c) an expression cassette for a bacterial selection marker, and
(d) a bacterial plasmid origin of replication,
wherein the first and second pA signals are the same or different and are selected from the group consisting of a thymidine kinase pA (TKpA) sequence and a simian virus 40 (SV40) early pA sequence,
wherein the first promoter is a cytomegalovirus (CMV) promoter construct that is at least 90% identical to nucleotides 69 to 1,716 of SEQ ID NO:1 or an Elongation factor 1-alpha (EF-1 alpha) promoter construct that is at least 90% identical to nucleotides 12-1,444 of SEQ ID NO:2;
wherein the second promoter is a 3-phosphoglycerate kinase (PGK) promoter if the eukaryotic selection marker is a puromycin resistance protein;
wherein the second promoter is a simian virus 40 (SV40) late promoter if the eukaryotic selection marker is a GS protein; and wherein the eukaryotic selection marker is a puromycin resistance protein if the first promoter is the CMV promoter.
